(19) United States
(12) Patent Application Publication
(10) Pub. No.: US 2006/0229344 A1
(43) Pub. Date: Oct. 12, 2006

(54) Method of treating amyotrophic lateral sclerosis

(76) Inventor: John Ravits, Mercer Island, WA (US)

Correspondence Address: JEFFREY B. OSTER, 8339 SE 57TH ST, MERCER ISLAND, WA, 98040, US

(21) Appl. No.: 11/101,791

(22) Filed: Apr. 7, 2005

(51) Int. Cl. A61K 31/4245

(52) U.S. Cl. 514/364

(57) ABSTRACT

There is disclosed a method for treating Amyotrophic Lateral Sclerosis (ALS), comprising administering an effective amount of an anti-viral agent. Specifically, the anti-viral agent is directed to RNA type viruses. More specifically, the anti-viral agent is pleconaril and pharmacologically acceptable salts to treat ALS and other neurodegenerative diseases.

### TECHNICAL FIELD OF THE INVENTION

The present invention provides a method for treating amyotrophic lateral sclerosis (ALS), comprising administering an effective amount of an anti-viral agent. Specifically, the anti-viral agent is directed to RNA type viruses and particularly pico RNA viruses. More specifically, the anti-viral agent are [(oxazolylphenoxy)alkyl]isoxazole capsid-binding compounds) including pleconaril and pharmacologically acceptable salts and are directed against picornavirus capsids.

### BACKGROUND OF THE INVENTION

ALS, also known as Lou Gehrig's disease, is a progressive, fatal neurodegenerative disorder causing degeneration of the motor neurons of the cortex, brain stem, and spinal cord. It produces progressive weakness of voluntary muscles and eventual death. The onset of disease is usually in the fourth or fifth decade of life, and most affected individuals succumb within 2 to 5 years of disease onset. ALS occurs in both sporadic and familial forms. Of the 5-10% of all cases that are familial (familial ALS or FALS), 20% carry a mutation of the superoxide dismutase 1 (SODI) gene that codes the ubiquitously expressed Cu,Zn-SOD enzyme. The etiology of sporadic ALS (SALS) is unknown.

Fundamental pathophysiological mechanisms of degeneration are not understood. Five major mechanisms and/or areas of interest are discussed in ALS although none of these have strong consensus. (A) Mutations of the SODI gene cause a poorly understood toxic gain of function; while mutations in the superoxide dismutase 1 (SOD 1) gene suggests that abnormal enzyme functioning may play a pivotal role in pathogenesis, in fact altered enzymatic function is probably not causative and instead the enzyme protein acquires some poorly understood toxic property. (B) Increased generation of oxygen free radicals, especially hydroxyl radicals known as “reactive oxygen species” (ROS), whose production exceeds the capacity of cellular mechanisms to remove/inactivate it, a condition known as “oxidative stress”. (C) Abnormalities in neurofilaments and cytoskeletal proteins perhaps by oxidative modifications or hyperphosphorylation may lead to selective motor axon degeneration. (D) Excitotoxicity caused by increased glutamate excitation together with alterations of excitatory amino acid transporters. (E) Abnormalities of mitochondrial function may lead to selective motor neuron failure perhaps by dysfunctioning energy production pathways in mitochondria, resulting in an excessive generation of damaging factors, including ROS. Whatever upstream mechanisms trigger degeneration, it appears that the final common pathway of motor neuron death is apoptosis (Guégan, J Clin. Invest. 111:153-161, 2003).

There is no promising treatment available to date. The only compound yielding borderline significance with respect to survival time is RILUZOLE (2-amino-6-(trifluoromethoxy) benzothiazole), an antiexcitotoxin (Rowland, New Engl. J. Med. 344:1688-1700, 2001). As much attention and research has been devoted to oxidative stress mediated by reactive nitrogen/oxygen species, new attempts of treatment are focusing on antioxidant strategies involving suppression of nitric oxide (NO) synthase. Apoptosis itself is a focus for therapies and current drugs such as minocycline that has anti-apoptotic properties and others in development are under development or investigation.

Accordingly, there remains a prominent need for greater understanding of disease mechanism and for new therapies preventing, slowing, or improving the loss of motor function in ALS patients. The present invention was made to address that need.

### SUMMARY OF THE INVENTION

The present invention posits a viral hypothesis as the underlying cause and pathology of ALS. Therefore, the viral hypothesis of ALS provides that the continual long-term administration of anti-picornaviral agents, particularly called capsid-binding compounds that stabilize capsids and prevent genome release, are effective for treating ALS.

Specifically, the present invention provides a method for treating ALS and other neurodegenerative diseases caused by picornaviruses, comprising administering an effective amount of an oxadiazolyl-phenoxyalkylisoxazole composition or a pharmaceutically acceptable salt thereof. Preferably, the oxadiazolyl-phenoxyalkylisoxazole agent is an [(oxazolylphenoxy)alkyl]isoxazole capsid-binding compounds or pleconaril. Preferably, the other neurodegenerative diseases are selected from the group consisting of ALS, Parkinson's disease, Alzheimer's disease, and multiple sclerosis. Most preferably, the neurodegenerative disease is ALS.

### BRIEF DESCRIPTION OF THE DRAWINGS

FIG. 1 shows the structure of the poliovirion as a complete capsid structure of virulent poliovirus type 1 [PV1(M)] illustrated as a water-accessible molecular surface. One of the 12 pentameric subunits of the capsid and its five constituent triangular pseudoprotomeric subunits are illustrated. The 5x and 3x labels indicate the locations of the fivefold and the threefold axes of this pentamer. The twofold axes occur at the intersection of the three adjacent pentamers. The central pseudoprotomer illustrates the subunit geometry of viral capsid proteins VP1, VP2, and VP3(ii). The biologically relevant protomer (to viral assembly) is pear-shaped and consists of VP1, VP2, and VP3(i). The internal VP4 protein is not visible from the surface. The canyon's north wall (A), south wall (C), and bottom (B) are indicated. The major poliovirus antigenic sites are labeled Ia, Ib, II, and III on an adjacent pseudoprotomer. (Harber et al., Canyon Rim Residues, Including Antigenic Determinants, Modulate Serotype-Specific Binding of Polioviruses to Mutants of the Poliovirus Receptor. Virology 1995, 214:559-570).

FIG. 2 shows locations of human PVR (hPVR) binding mutations on the poliovirus capsid and a virus-receptor model. FIG. 2(a) is a stereo view showing details of the fivefold depression, referred to as the canyon. The axes of icosahedral symmetry are labeled around a single representative of the 60 triangular pseudoprotomeric facets. The view is seen along the icosahedral twofold axes of symmetry looking down upon the canyon area. Residues exchanged in the antigenic hybrids (NAgI and NAgII) are represented in magenta color, while the amino acid substitutions resulting from site-directed mutagenesis are colored in cyan. The sphingosine molecule occupying the hydrophobic pocket of the viral capsid protein VP1 protein is shown in yellow. FIG. 2(b) is a stereo view of the same area as that shown in (a) except that the view is perpendicular to the axes of the icosahedral five- and twofold symmetry. FIG. 2(c) is a poliovirus receptor modeled after the CD4 molecule is docked into the canyon. Orientation is the same as that in (b). Domain 1, an immunoglobulin V-like domain (gold) enters the canyon. The smaller domain 2, an immunoglobulin C-like domain, is colored green and sits above the surface of the virion. It is possible that domain 1 contacts residues of the north wall (nearest NAgI) and south wall (the NAgII face) simultaneously based on spatial considerations alone. Also, binding of the receptor to the canyon rim regions does not necessarily involve contacts of the receptor to the bottom of the canyon. (Harber et al., Canyon Rim Residues, Including Antigenic Determinants, Modulate Serotype-Specific Binding of Polioviruses to Mutants of the Poliovirus Receptor. Virology 1995; 214:559-570.)

FIG. 3 shows a diagrammatic view of picornavirus with enlargement of one icosahedral asymmetric unit showing the outline of the canyon and the entrance to the antiviral-binding pocket. The protomeric assembly unit (which differs from the geometric definition of the asymmetric unit) is shown in heavy outline on the icosahedron. (Oliveira et al., Structure 1993; 1:51-68.)

### DETAILED DESCRIPTION OF THE INVENTION AND RATIONALE OF APPLICATION

At present the most preferred compound is pleconaril or a derivative thereof. Pleconaril: (3-[3,5-dimethyl-4-[[3-(3-methyl-5-isoxazolyl)propyl]oxy]phenyl]-5-(trifluoromethyl)-1,2,4-oxadiazole) (ViroPharma Inc.; Picovir(®). Compounds comprising Pleconaril and derivatives thereof are shown in U.S. Pat. No. 5,464,848, the European equivalent EP 0566199, and are claimed to have effect on picornaviral infections. Other derivatives of Pleconaril claimed to have antiviral effects against picornavirus are disclosed in U.S. Pat. No. 4,945,164. There are numerous antiviral compounds effective against picornaviruses disclosed in the prior art with several different modes of actions. Such compounds are, for example, compounds that inhibit the proteolytic activity of picornaviral proteases disclosed in the patent application WO 9222570; 2-(4-pyridylaminomethyl) benzimidazole derivatives with in vitro and in vivo antipicornavirus activities disclosed in EP 0252507B1. Benzisoxazole derivatives for treatment of picornavirus infection are disclosed in WO 0250045. (All the mentioned references are incorporated herein by reference.)

Pharmaceutical Formulations

While it is possible that an antiviral compound may be administered as the neat chemical, it is preferable to present the active ingredient as a pharmaceutical formulation or as a medicament. A suitable medicament or pharmaceutical formulation useful in the present invention comprises an antiviral compound together with one or more pharmaceutically acceptable carriers and, optionally, other therapeutic and/or prophylactic ingredients. The carrier (s) must be acceptable in the sense that it should be compatible with the other active or inactive ingredients of the formulation and not deleterious to the recipient thereof. The antiviral compounds used in the invention may also be used in combination with other anti-viral agents or other pharmaceuticals used in the treatment of viral infections. Representative examples of other active ingredients include immunomodulators, immunostimulants, such as various interleukins, cytokines and antibody preparations, antibiotics and anti-inflammatory agents.

Pharmaceutical formulations include those suitable for oral, rectal, nasal, topical (including buccal and sub-lingual), vaginal or parenteral (including intramuscular, subcutaneous and intravenous) administration or in a form suitable for administration by inhalation or insufflation. The compounds of the invention, together with a conventional adjuvant, carrier, or diluent, may thus be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

Viral Hypothesis—Background and Summary

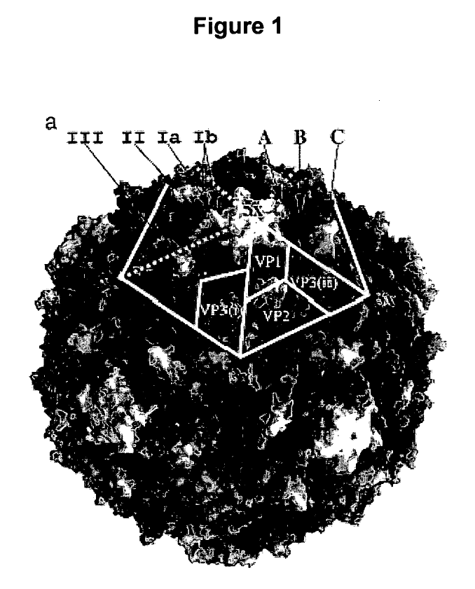

A viral cause of SALS has been hypothesized (reviewed in Salazar-Gureso and Roos, Clin. Neurosci. 1995;3(6):360-7; Rowland and Shneider, New Engl. J. Med. 2001; 344:1688-1700; Viola et al., Ann. Neurol. 1979;5:402-403; Cremer et al., Arch. Neurol. 1973;29:331-333; Muller and Schaltenbrtand, J. Neurol. 1979;220:1-19; Norris, Neuro. Neuro. Psychiatr. 1977;18(2-3 sppl):443-454; Weiner et al., Neurology 1980:30:1319-1322; Gibbs and Gajdusek, In: Rowland (ed), Human Motor Neuron Diseases. New York: Raven Press, 1982: pp 343-353; Fallis and Weiner, In: Rowland (ed), Human Motor Neuron Diseases. New York:Raven Press, 1982:355-361; Kennedy, J. Royal Soc. Med. 1990;83:784-787; Jubelt, Adv. Neurol. 1991;56:463-472; Fraser et al., Brain Pathology 1996;6:89-100; Miller et al., Neurology 1980;30:884-886; Kohne et al., J. Gen. Virol. 1981;56:223-233; Brachic et al., Ann. Neurol. 1985;18:337-343; Love, Brain Pathol. 1996;6:99-100; and Muir et al., J. Gen. Virol. 1996, 77:1469-1476). Enteroviruses (FIG. 1) have led the candidate viruses because of the tropism of poliovirus, an enterovirus subtype, for motor neurons. Retroviruses are also candidates because motor neuron syndromes are associated with both HIV and HTLV1 retroviral subtypes. To date, three studies from two laboratories have reported evidence of enterovirus in nervous systems of patients dying from SALS using RT-PCR and three studies from three laboratories, two of them recent, have reported negative results with this technique.

Viral persistence is non-lytic and non-cytopathic infection that evades host's immune surveillance. Viral properties, host susceptibility factors, and time of exposure may all be important factors in its establishment. Apoptosis, a major factor in motor neuron death in SALS, is a process of active non-necrotic cell death that has complex interplay with viruses and may be either promoted or opposed by them. Viral tropism is the process by which viruses select and propagate to target cells. It is controlled by capsid conformation and surface receptors on host cells. Picornaviruses in general and enteroviruses in particular have a region on their capsids known as the canyon which docks on cell receptors. Docking induces conformational changes of the capsid and genome release. Poliovirus, for example, is tropic for motor neurons by docking on poliovirus receptor. It penetrates the motor system focally after crossing either the blood-muscle or the blood-brain barriers. It propagates bidirectionally along axons and synapses to contiguous motor neurons, upper as well as lower, which sequester infection and create avenues for spread over long distances. If chronic and persistent rather than acute and lytic, such viruses trafficking in a finite system of non-dividing cells and inducing apoptosis would cause cell death that summates linearly rather than exponentially. Taken together, these mechanisms explain signature clinical features of SALS—focal onset weakness, contiguous or regional spread of weakness, confinement to upper and lower motor neurons, and linear rates of progression. This hypothesis predicts that continual long-term administration of anti-picornaviral agents called capsid-binding compounds which stabilize capsids and prevent genome release would be efficacious in ALS.

Viral Persistence (Non-Lytic Infection) and the Privilege of the CNS

Viral persistence is non-lytic and non-cytopathic infection that evades host's immune surveillance. Host as well as viral factors are significant since some viruses cause lytic infection in one cell line and persistent infection in others. Time of infection is also significant since infection causes lytic infection at one time in the life of the host and persistent infection at another. Viruses escape immune surveillance to persist in host cells through a number of strategies. Enteroviruses can cause persistent as well as lytic infections. Polioviruses, in particular, can cause persisting infection. The central nervous system (CNS) is a unique compartment for persisting infection: it is relatively isolated from the immune system by the blood-brain barrier, its neurons have relative absence of major histocompatibility-complex molecules fundamental to invoking immunologic response, its cells are static and cannot be overgrown by replacements, and its extensive networks of axons and dendrites create avenues for sequestering and spreading infection over long distances. SALS is fundamentally a disease of the CNS—all motor neurons reside inside the CNS compartment and only axons of the lower motor neurons extend outside it. SALS, traditionally regarded as a disease in which inflammation and immune response are absent, in fact, has subtle responses.

Viral-Associated Apoptosis

The interplay of viruses and apoptosis, a process of active non-necrotic cell death, have been speculated but never proven in neural degenerations. Apoptosis has complex interplay with viruses and may be either promoted or opposed by them. One example is the poliovirus, which can either induce or oppose apoptosis depending on viral properties and host cell factors. Apoptotic capability is clearly established in motor neurons and it could be pathogenically activated by viral infection in ALS as the ultimate mechanism of cell death.

Cell Surface Receptors, Viral Tropism, and Viral Sequestration.

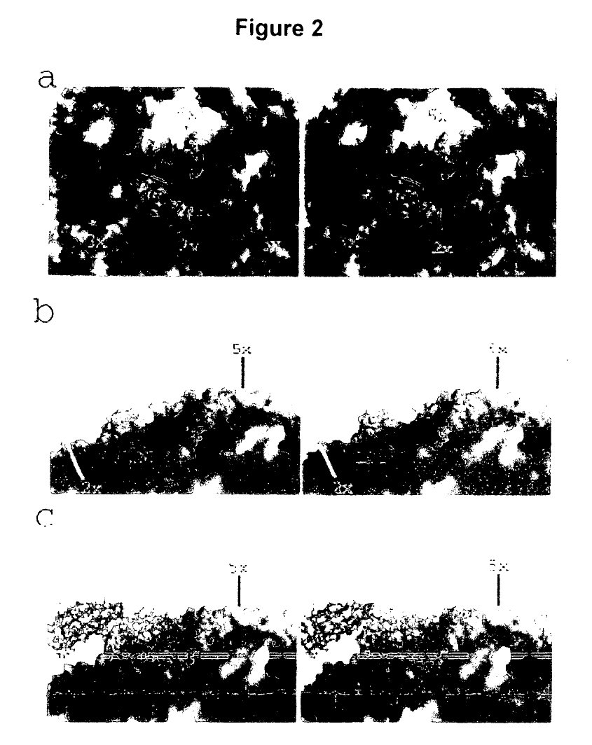

Viral tropism is the process by which viruses select and propagate to targets cells. It is controlled by capsid conformation and receptors on the cell's surface. Models are the picornaviruses, especially polioviruses, which are tropic for motor neurons. Key to poliovirus's tropism is the poliovirus receptor (PVR). PVR underlies both viral selection and infection—PVR protruding from the cell surface fits into a depression on the surface of the virion capsid known as the canyon, a depression located just below the fivefold axis of symmetry on the north face of the icosahedal structure (FIG. 2). This induces conformational changes in the capsid leading to destabilization, uncoating and RNA release.

Focal Access and Contiguous Viral Propagation.

One of the best-understood and most relevant models of viral propagation is again the poliovirus. Extensive clinical and experimental observations indicate poliovirus penetrates the motor system either from the periphery or from the CNS. From the periphery, it establishes a focal nidus in muscle by first crossing the blood-muscle barrier and then travels retrograde along motor neurons to invade the motor system. From the CNS, it first crosses the blood-brain barrier then invades the motor system. Once inside motor neurons, poliovirus propagation is trans-neuronal to contiguous motor neurons, either horizontal between neighboring neurons or trans-synaptic between upper and lower motor neurons. Motor neurons create avenues for spreading over long distances infection already sequestered by its unique tropism. Thus, poliovirus infection begins focally and spreads contiguously to infect the entire motor system, upper as well as lower motor neurons.

Viral Etio-Pathogenesis of SALS

Cardinal clinical features of SALS are focal onset, regional or contiguous spread, confinement to upper and lower motor neurons, relatively linear progression for each patient but highly variable among different patients. These features are readily explained by persistent viral infection. Persistent infection is non-lytic and non-cytopathic. Viral properties, host susceptibility factors and time of exposure may all be important factors in its establishment. Tropism for motor neurons, controlled by viral capsid conformation and host cell-surface receptors, ensures infection selects motor neurons and stays sequestered. Infection may gain access either after penetrating the blood-muscle barrier or the blood-brain barrier. Once inside, it spreads contiguously. Horizontal propagation includes crossing the midline at spinal and brainstem levels because of relative proximity of anterior horns and brainstem nuclei. Vertical or trans-synaptic propagation causes jump between lower and upper motor neurons, the latter leading to propagation over long distances. Because infection is persistent and propagation through the motor system is successive, progression is linear rather than accelerating. Because of variation of biologic factors such as viral load, viral virulence and host cell factors, progression rates are highly variable among different host patients. As infection propagates, it switches on apoptosis, a capability well established in motor neurons and known to have complex interplay with viruses. Since motor neurons are limited in number and non-dividing, cell death summates. This is manifested clinically as progressive muscle weakness that begins focally, spreads regionally, and progresses linearly.

Implications for Therapy

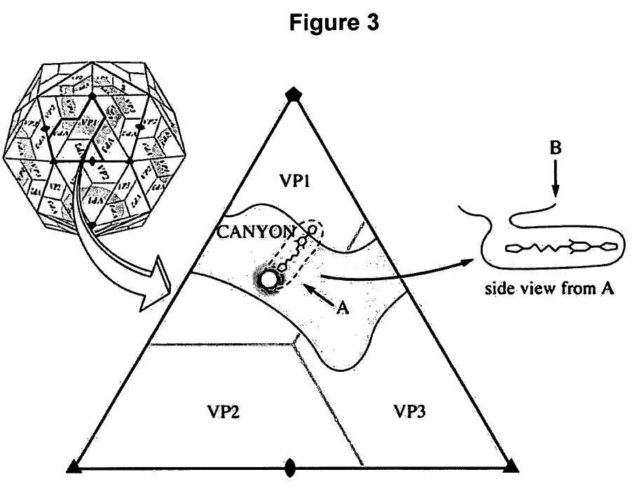

1. Capsid-Binding Compounds. The capsid binding compounds, [(oxazolylphenoxy)alkyl]isoxazoles, have been designed for treatment of enteroviral and Picornavirus infections. These compounds bind in the hydrophobic pocket situated at the base of the canyon site on the north face of the virion icosahedral capsid where cellular receptors interact. This binding raises the floor of the canyon and alters the virion's ability to attach and bind to receptors, thus inhibiting disassembly and RNA release (FIG. 3). Since these drugs act on the virion's capsid and not its RNA genome, they block viral propagation-once virus has released RNA and infected a cell, capsid-binding drugs would have little effect. Therefore, for them to be effective in SALS, assuming the viral pathogenesis, a constant level of drug would have to be present in the body over months or years and it would be controlling rather than curative treatment. Since the hydrophobic pocket has unique topology not found in other classes of proteins, the compounds are viral specific and have minimal host toxicity (166). One of these compounds, VP 63843 (Pleconaril®), is well tolerated, well absorbed after oral administration, has few side-effects, little toxicity, and crosses readily into the central nervous system and into gray matter.

2. Other Therapeutic Strategies. Other specific anti-picornavirus therapy includes inhibitors of 3C protease and recombinant soluble intercellular adhesion molecules. Interferons are potent mediators resisting viruses. Cells exposed to viruses increase production of interferon through an intracellular cascade and this increases resistance to infection. Alpha interferon, a potent inhibitor of picornaviral infection, may be even more effective when combined with capsid-binding compounds. Therapeutic strategies to alter apoptosis pathways are in early stages of development. Strategies combining antiviral and anti-apoptotic with current anti-neuroexcitatory therapies may be effective.

Mechanisms and Predictions

Without being bound by theory, the following mechanisms are predicted: 1) viral presence may be detectable only using highly sensitive techniques on optimally procured and processed nervous system tissue. Laser microdissection is a new technology that allows isolation of cells and thus will overcome problems of sampling. 2) Host genomic factors such as cell surface receptors or host immune factors may be important susceptibility factors that permit establishment of persistent infection. 3) ALS disease may depend on the chance concurrence of these genetic susceptibility factors, sporadic infection, motor system penetration provided through the blood-muscle barrier breakdown (from exercise, trauma or other factors), and possibly vulnerable times in the host's development. 4) An animal model might be created by inserting genetic susceptibility factors such as cell surface receptors into a transgenic animal and inoculating with biologically viable extract from motor neurons—intramuscular inoculation may allow better entry to the motor system than intracerebral inoculation. Controlling times of exposure may be important. And 5) continual long-term administration of capsid-binding compounds such as Pleconaril that readily penetrates the CNS may be efficacious.

I claim:

1. A method for treating ALS and other neurodegenerative diseases caused by Picornaviruses, comprising administering an effective amount of an oxadiazolyl-phenoxyalkylisoxazole composition or a pharmaceutically acceptable salt thereof.

2. The method of claim 1 wherein the oxadiazolyl-phenoxyalkylisoxazole agent is a [(oxazolylphenoxy)alkyl]isoxazole capsid-binding compounds or pleconaril.

3. The method of claim 1 wherein the other neurodegenerative diseases are selected from the group consisting of ALS, Parkinson's Disease, Alzheimer's Disease, and multiple sclerosis.

4. The method of claim 3 wherein the other neurodegenerative diseases is ALS.
